(19) United States
(12) Patent Application Publication
(10) Pub. No.: US 2026/0198840 A1
(43) Pub. Date: Jul. 16, 2026

(54) MULTIPLE WELL EPICUTANEOUS TEST PATCH ARRAY

(71) Applicant: Immunoderm Inc., Edmonton (CA)

(72) Inventor: John Elliott, Edmonton (CA)

(21) Appl. No.: 19/135,560

(22) PCT Filed: Dec. 20, 2023
(86) PCT No.: PCT/CA2023/051714

§ 371 Date: Jun. 4, 2025

(51) Int. Cl. A61B 5/00

(52) U.S. Cl. CPC A61B 5/411; A61B 5/6833; A61B 5/68335

(57) ABSTRACT

The present invention provides for a novel epicutaneous test plaster useful for the application of liquid test substances for direct and continuous contact with the skin of a human patient. Further provided is a device useful for the sealing of the epicutaneous test plaster of the present invention, which enables the storage and transport of said test plasters preloaded with liquid test substances, prior to their application to the skin of a human patient.

WO

### FIELD OF THE INVENTION

The present invention pertains to an application aid useful for assessment of dermatological sensitivities or allergic responses to haptens in gel or liquid vehicles through use of an epicutaneous test patch array and devices for sealing same.

### BACKGROUND OF THE INVENTION

All of the publications, patents and patent applications cited within this application are herein incorporated by reference in their entirety to the same extent as if the disclosure of each individual publication, patent application or patent was specifically and individually indicated to be incorporated by reference in its entirety.

Epicutaneous plasters (also referred to as test patches or test plasters) are commonly used for testing of patients for allergies or sensitivities to compounds, such as known allergens or haptens; in which a series of suspected haptens are loaded onto the epicutaneous plaster, which is then attached to the skin of a patients for up to 7 days; following which the plaster is removed and the skin of the patient observed for irritation, inflammation or other reactions associated with allergies or sensitivities. As well as needing to maintain separation between the various different haptens being tested on a patient, the epicutaneous patch must also allow flexibility, in order to maintain contact between the loaded hapten and the patient's skin. Further, it has become common in clinical practice to test at least 40 haptens concurrently; and therefore, during the hapten loading process it can be an inconvenience for the clinical practitioner to employ epicutaneous plasters with 12 or fewer chambers per plaster.

The art describes test plasters, or test patches, useful for providing continuous contact of a test substance to selected areas of skin of a patient. For example, U.S. Pat. No. 7,798,976 describes an epicutaneous test plaster with a plurality of test chambers, each test chamber comprised of a support element secured to a carrier, a carrier, a frame shaped foam plastic lattice and a second lattice of adhesive interposed between the foam plastic and support element.

Although useful for maintaining contact between a test substance (for example a hapten) and the skin, prior art devices suffer from limitations; for example, in identifying or correlating chambers with the hapten loaded into the chamber once applied to the skin. In this regard prior art epicutaneous test plasters generally require markings to be made on the patient's skin at the periphery of the rectangular patch, and after removal of the patch these peripheral markings are used to orient where the various test chambers were originally located on the patient's skin. In contrast the epicutaneous test plaster described by the present inventors in U.S. Pat. No. 11,020,044 has central holes which can be marked to more efficiently and precisely orient where the various test chambers were originally located on the patient's skin. By way of another exemplary limitation, prior art devices are difficult to handle once the support element is removed, such as during loading. By way of another exemplary limitation, the epicutaneous plasters, due to their design, have limitations to their size and/or number of separated chambers they may contain. This is a function of the volume of hapten generally used in clinical practise, the orientation and ordering of the chambers within the epicutaneous plaster, and the use of wells made from metal or stiff plastic which limits the ability to maintain adhesion of the chamber to the skin during movement of the patient.

While the prior art contemplates epicutaneous plasters capable of maintaining contact with the skin and the test substance; the art has suffered from the inability to provide epicutaneous plasters capable of receiving liquid. While the epicutaneous plasters of the prior art were capable of receiving a liquid hapten, the epicutaneous plasters would need to be applied to the patient quickly so as to avoid evaporation of the liquid hapten or the solvent of a hapten. Further, application of the epicutaneous plasters was difficult, with the potential for the liquid within the chambers to spill.

More deleterious, was the spreading of the liquid hapten from one chamber to areas surrounding the chamber, which provided the opportunity for cross-contamination between test chambers, confounding the ability of a health professional to assess the results of the hapten's interaction with the patient's skin.

Further, the storage and transport of liquid haptens within the test chambers of prior art epicutaneous plasters is hindered by evaporation, or the spilling of the liquid from the test chambers prior to application to the patient or after. The prior art provides for sealing of epicutaneous plasters containing a hapten, intended for storage or transport, for example by way of a reversible cover layer as described in U.S. Pat. No. 11,020,044. While providing sufficient protection of the chambers for storage and transport absent haptens, or with haptens distributed through a viscous fluid (i.e. gel) such as petroleum jelly; the reversible cover layer described in the prior art is not suited for storage and transport of liquids or fluids within the chamber.

The art is in need of an epicutaneous plaster that allows improved loading of liquid haptens into the plaster, improved handling during and following loading of the liquid haptens, reduction of cross-contamination of liquid haptens between the separated chambers, improved storage and transport and allows for the implementation of larger number of test chambers on a contiguous plaster. Further, the art is in need of in improved means to seal an epicutaneous plaster containing haptens, liquid or otherwise, for improved storage and transportation.

### SUMMARY OF THE INVENTION

In one aspect the present invention provides for an epicutaneous test plaster comprising a support lattice comprised of a material impermeable to liquid and gas containing a first multiplicity of holes, a flexible carrier lattice containing a second multiplicity of holes, a first adhesive layer for removable adhesion of the epicutaneous plaster to a skin portion, a second adhesive layer for binding the support lattice to the flexible carrier lattice, a liquid and gas impermeable cover layer extending over all the first adhesive layer of said flexible carrier lattice, and a plurality of fluid absorbent material distributed over the support lattice; wherein the second multiplicity of holes on the flexible carrier lattice is greater than the first multiplicity of holes on the support lattice; wherein said flexible carrier lattice is adhered to the support lattice by way of the second adhesive layer; wherein the plurality of test chambers are formed by way of second multiplicity of holes in the flexible carrier lattice describing a frame around a contiguous portion of the support lattice; wherein a subset of the first multiplicity of holes on the support lattice align with the second multiplicity of holes in the flexible carrier lattice; wherein the liquid and gas impermeable cover layer forms a seal over the top of the plurality of test chambers; wherein the fluid absorbent material is located within said plurality of test chambers, said fluid absorbent material not in contact with either of said flexible carrier lattice or liquid and gas impermeable cover layer; and wherein the liquid and gas impermeable cover layer is removably secured to the flexible carrier lattice by way of said first adhesive layer. In one embodiment the flexible carrier lattice is comprised of polyethylene foam and said first and second adhesive layers are comprised of a medical grade adhesive. In a further embodiment the polyethylene foam has a thickness of between 0.4 mm and 0.6 mm. In another embodiment said liquid and gas impermeable layer is a lattice with a third multiplicity of holes. In a further embodiment said third multiplicity of holes align with the second multiplicity of holes in the flexible carrier lattice. In another embodiment at least one unique chamber identifier is printed on the material impermeable to liquid and gas proximate to a test chamber. In a further embodiment the liquid and gas impermeable layer is low density polyethylene of 2 mil thickness. In another embodiment the liquid and gas impermeable cover layer extends above the plane of the first adhesive layer over the test chamber.

The present invention also provides for a sealing tool for use in sealing an epicutaneous test plaster of the present invention the sealing tool comprising a lower portion with a multiplicity of lower openings, and an upper portion with a multiplicity of upper openings; wherein said lower portion is recessed so as to receive said epicutaneous patch; wherein said upper portion protrudes an amount equivalent to the recession of said lower portion; wherein both upper portion and lower portion mirror the shape of the epicutaneous test patch such that said lower portion can receive said upper portion; and wherein lower portion lower openings and upper portion upper openings are positioned such that when said epicutaneous test patch is placed within said lower portion the chambers in said epicutaneous patch are aligned with both the upper portion upper openings and lower portion lower openings. In one embodiment the upper portion and lower portion are in mechanical communication by way of a hinge.

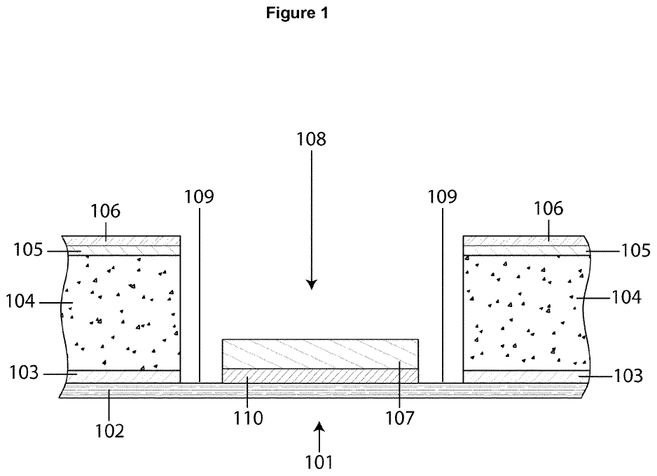

In one aspect the present invention provides for an epicutaneous test plaster comprising a support lattice comprised of a material impermeable to liquid and gas containing a first multiplicity of holes, a flexible carrier lattice containing a second multiplicity of holes, a first adhesive layer for removable adhesion of the epicutaneous plaster to a skin portion, a second adhesive layer for binding the support lattice to the flexible carrier lattice, a liquid and gas impermeable cover layer extending over all the first adhesive layer of said flexible carrier lattice, and a plurality of fluid absorbent material distributed over the support lattice; wherein the second multiplicity of holes on the flexible carrier lattice is greater than the first multiplicity of holes on the support lattice; wherein said flexible carrier lattice is adhered to the support lattice by way of the second adhesive layer; wherein the plurality of test chambers are formed by way of second multiplicity of holes in the flexible carrier lattice describing a frame around a contiguous portion of the support lattice; wherein a subset of the first multiplicity of holes on the support lattice align with the second multiplicity of holes in the flexible carrier lattice; said fluid absorbent material not in contact with said flexible carrier lattice; wherein the liquid and gas impermeable cover layer forms a seal over the top of the plurality of test chambers; wherein the fluid absorbent material is located within said plurality of test chambers; and wherein the liquid and gas impermeable cover layer is removably secured to the flexible carrier lattice by way of said first adhesive layer. In one embodiment the flexible carrier lattice is comprised of polyethylene foam and said first and second adhesive layers are comprised of a medical grade adhesive. In a further embodiment the polyethylene foam has a thickness of between 0.4 mm and 0.6 mm. In another embodiment said liquid and gas impermeable layer is a lattice with a third multiplicity of holes. In a further embodiment said third multiplicity of holes align with the second multiplicity of holes in the flexible carrier lattice. In another embodiment at least one unique chamber identifier is printed on the material impermeable to liquid and gas proximate to a test chamber. In a further embodiment the liquid and gas impermeable layer is low density polyethylene of 2 mil thickness BRIEF DESCRIPTION OF THE FIGURES FIG. 1 shows a cross section through a contiguous portion of a preferred embodiment of the present invention, prior to loading with a liquid test substance (liquid hapten);

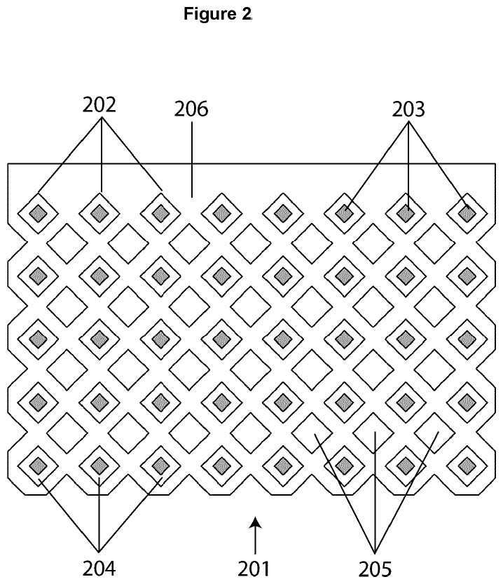

FIG. 2 shows a complete epicutaneous test patch of the present invention, with all layers shown in FIG. 1 present, prior to loading with liquid haptens;

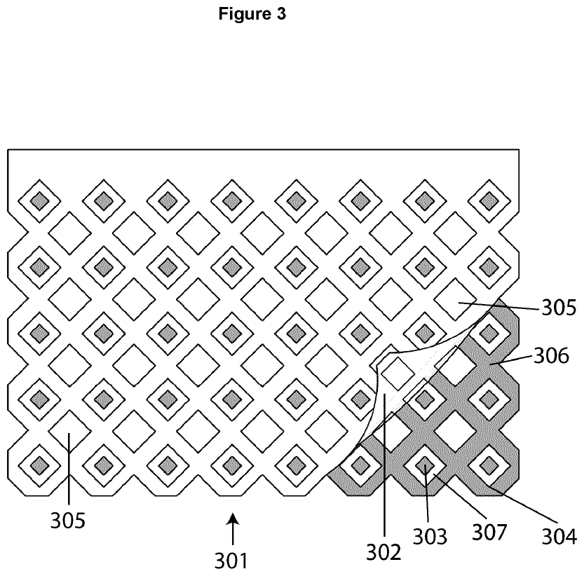

FIG. 3 shows an embodiment of an epicutaneous test patch of the present invention, with removable cover layer represented as being peeled from the patch after the patch has been loaded with liquid haptens;

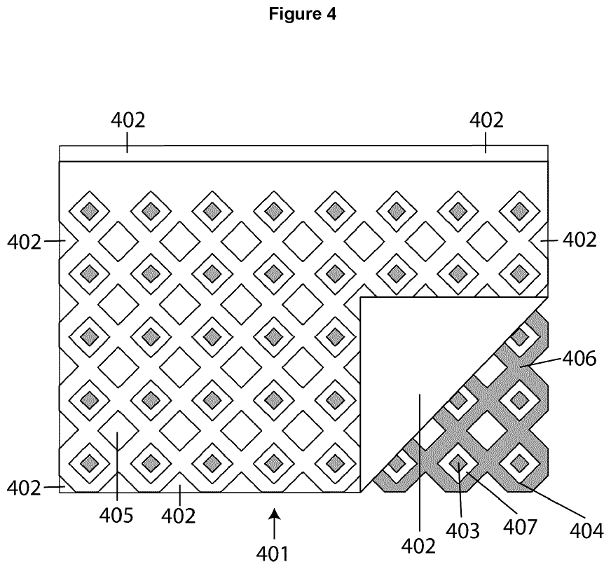

FIG. 4 shows one embodiment of the present invention, demonstrating an epicutaneous test patch previously loaded with liquid haptens with the liquid and gas impermeable cover layer, illustrated as transparent;

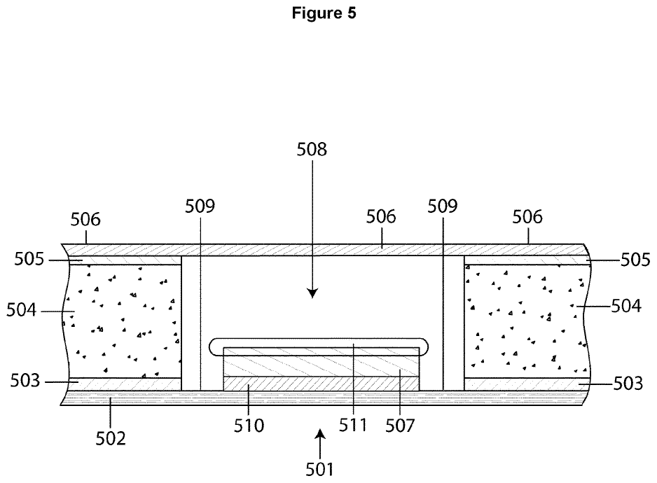

FIG. 5 shows a cross section through a contiguous portion of a preferred embodiment of the present invention with a liquid and gas impermeable cover layer in place;

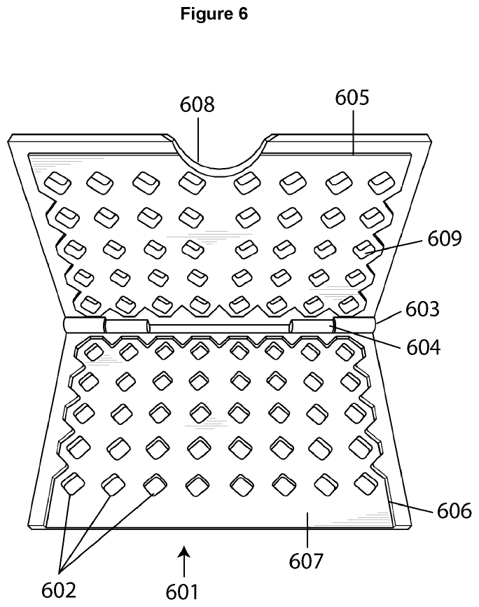

FIG. 6 shows a loading and sealing device of the present invention in a partially closed position;

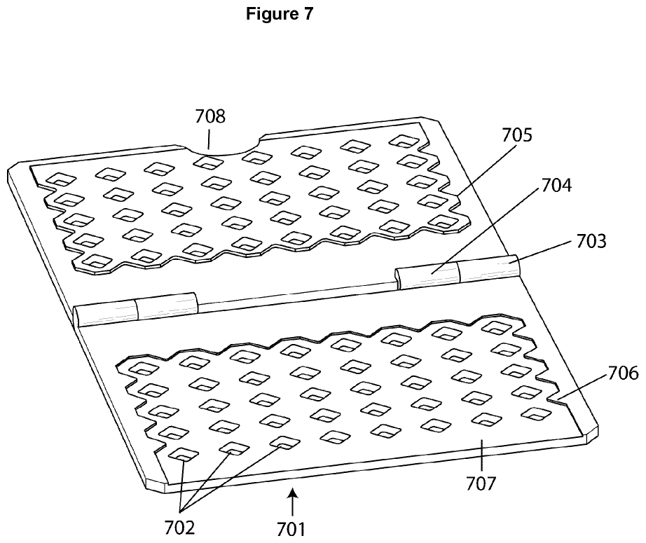

FIG. 7 shows a loading and sealing device of the present invention in a fully open position;

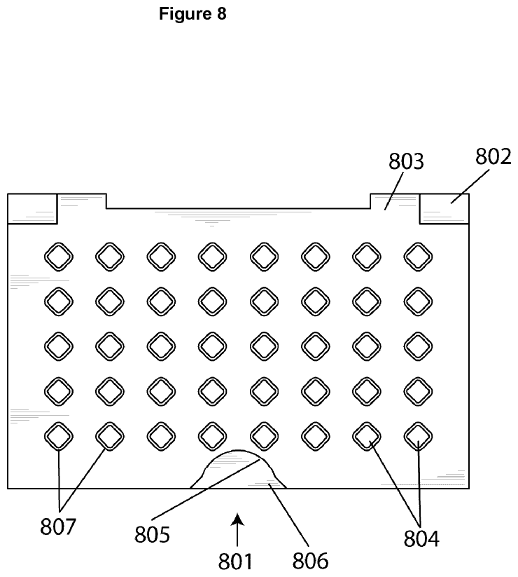

FIG. 8 shows a loading and sealing device of the present invention in a fully closed position;

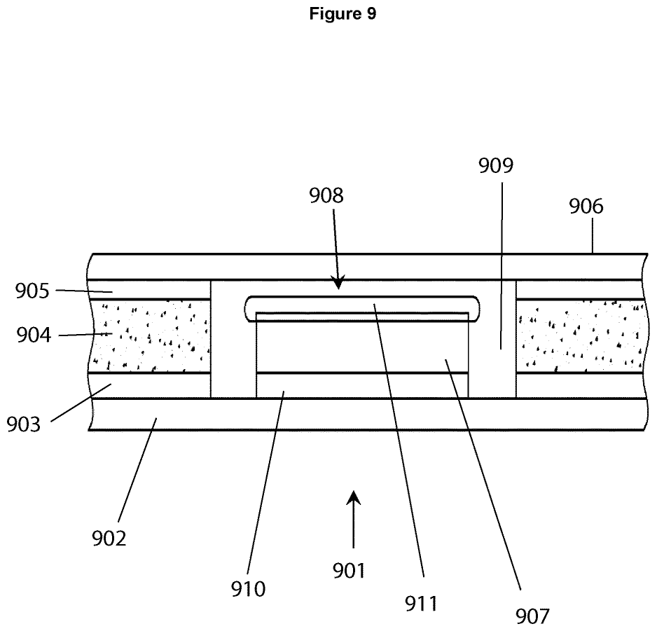

FIG. 9 shows a cross section through a contiguous portion of an embodiment of the present invention with a liquid and gas impermeable cover layer in place in close proximity to a fluid placed on the absorbent material;

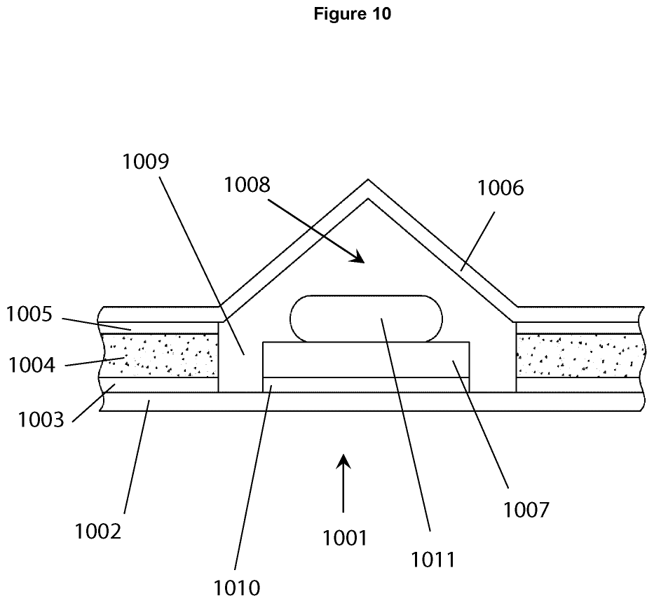

FIG. 10 shows a cross section through a contiguous portion of an embodiment of the present invention with an alternative embodiment of a liquid and gas impermeable cover layer in place;

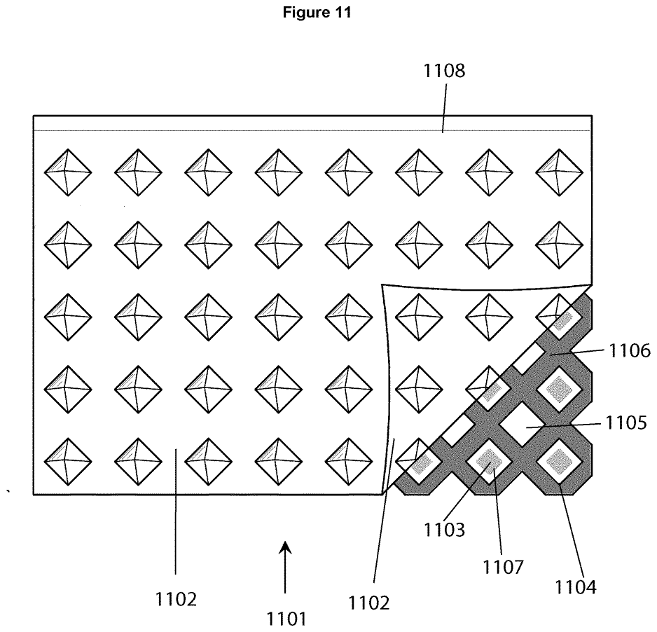

FIG. 11 shows an embodiment of the present invention, demonstrating an epicutaneous test patch previously loaded with liquid or gel haptens with an alternative embodiment of a liquid and gas impermeable cover layer; and

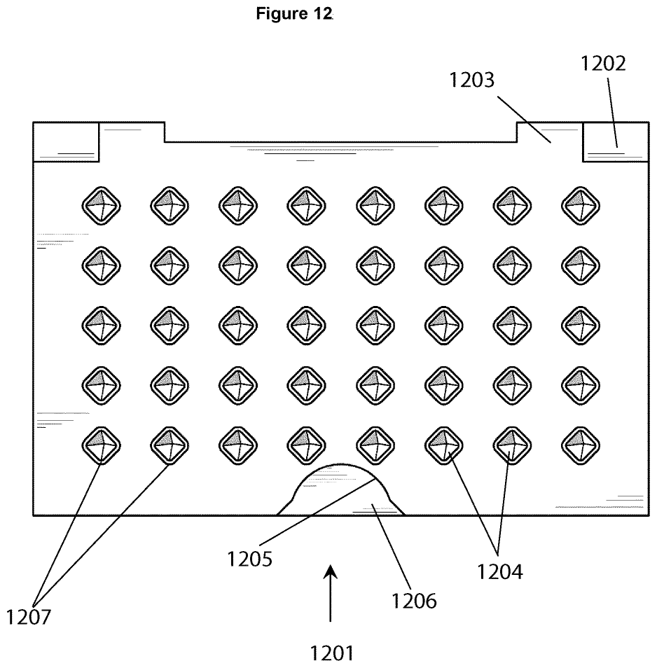

FIG. 12 shows a loading and sealing device of the present invention in a fully closed position with an alternative embodiment of a liquid and gas impermeable cover layer in place.

### DETAILED DESCRIPTION OF THE PRESENT INVENTION

According to the present invention, individual test chambers in an epicutaneous test plaster are formed by the joining of two lattices, a support lattice and a flexible carrier lattice, where the number of holes in the flexible carrier lattice are greater than the number of holes in the support lattice. The layering of the flexible carrier lattice overtop the support lattice results in the formation of wells formed with a base of the support lattice, and holes passing through both the support lattice and the flexible carrier lattice. The support lattice is comprised of a material which is impermeable to the liquid intended to be placed within the test chambers, which in the case of liquid haptens used for testing of allergies or sensitivities in human patients, is commonly water; thereby providing a moisture barrier on the underside of the test chamber.

Within each of the test chambers is located an absorbent material, in contact with the moisture barrier but not in contact with the support lattice, all as more fully described herein. It is contemplated that the removable cover layer may be a silicone release paper, comprised of a paper treated for improved sealing, improved water impermeability or optimal adhesion strength to the adhesive layers described further herein.

As used herein, “liquid haptens” means hapten preparations used in clinical practice that are in a liquid state at room temperature, which may arise from a solid, liquid, or gas being suspended or dissolved in a liquid carrier or solvent, which is typically water. As used herein “gel haptens” means hapten preparations used in clinical practice that are in a gel state at room temperature, which may arise from a solid, liquid, or gas being suspended or dissolved in a semi-solid gel carrier, which is typically petrolatum. As used herein the term “hapten” means a substance that after being compounded appropriately into liquid or gel form is capable of eliciting an immune response in testing of patients for allergies or sensitivities to compounds.

The present invention contemplates an epicutaneous test plaster for storage and transport formed by a flexible carrier lattice and a support lattice; the support lattice comprised of a contiguous layer of liquid and gas impermeable material such as polyethylene; the flexible carrier lattice comprised of two layers of adhesive with a polyethylene foam core interposed between the two; and the test plaster sealed by way of a liquid and gas impermeable removable cover layer placed over the entirety of the flexible carrier lattice on the side opposing the support lattice. The present invention further contemplates a device for promoting a seal between the liquid and gas impermeable cover layer and the flexible carrier lattice.

FIG. 1 shows a cross section of an individual test chamber 101 of an epicutaneous test plaster of the present invention, comprised of support lattice 102 which is a material impermeable to liquid and gas, and flexible carrier lattice 104 between adhesive layer 103 and 105. Removable cover layer 106 protects adhesive layer 105 until the epicutaneous test plaster is ready to be placed on the patients skin or sealed for storage or transport. Test chamber 108 is formed by flexible carrier lattice 104 and adhesive layers 103 and 105; and is bounded on the bottom by support lattice 102. Within test chamber 108 is absorbent material 107, which is not in contact with the flexible carrier lattice 104 or adhesive layer 103; the separation represented by 109. Absorbent material 107 is adhered to the support lattice 102 by way of adhesive 110, by way of non-limiting example 3M 1524 medical grade adhesive. In a preferred embodiment the adhesive is provided as a layer contiguous with absorbent material 107, as presented in FIG. 1 as 110. Selection of adhesive used for adhering absorbent material 107 to the flexible carrier lattice is contemplated to be based upon the type of liquid being placed on the absorbent material, the composition of the support lattice, interactions with human skin, and composition of absorbent material; selection of which are all within the capabilities of one skilled in the art. It is contemplated that removable cover layer 106 may be replaced by a contiguous liquid and gas impermeable cover layer following loading of a liquid hapten into chamber 108; said liquid and gas impermeable cover layer extending over, and sealing, chamber 108, for improved storage or transport, as further described herein. By way of non-limiting example the contiguous liquid and gas impermeable cover layer may be polyethylene film.

Adhesion of the support lattice to the flexible carrier lattice may be undertaken by means generally known in the art, but in a preferred embodiment an adhesive is applied as layer 103 which does not extend into test chamber 108. Adhesion of the support lattice to the removeable cover layer may be undertaken by means generally known in the art, but in a preferred embodiment an adhesive is applied as layer 105, the adhesive selected to have an adhesion strength greater with the flexible carrier lattice 104 than with the removable cover layer 106, and the adhesion strength between the adhesive and the flexible carrier lattice 104 and human skin (not shown) when the epicutaneous test plaster is placed on a patient. In a preferred embodiment, adhesive layers 103 and 105 are medical grade adhesives, for example methacrylate-based adhesives as known in the art. The adhesive used for layers 103 and 105 may be the same, the differential in the adhesion strength between support lattice 102 and flexible carrier lattice 104, and flexible carrier lattice 104 and removable cover layer 106; arising not from the adhesive used, but from the composition of the removable cover layer or treatment of the support lattice.

The composition of flexible carrier lattice is chosen to provide a balance between torsional flexibility of the epicutaneous test plaster on a patient, which is further improved by the holes formed between the support lattice and the flexible carrier lattice; with rigidity useful in the packaging, transport, and addition of haptens to the test chambers formed within the epicutaneous test plaster. Polyethylene foam of between 0.4 mm and 0.6 mm, more preferably 0.5 mm; has been found to provide the necessary characteristics, and in a preferred embodiment the flexible carrier lattice is formed by double-sided self-adhesive foam sold by Scapa group Plc, United Kingdom under catalogue number 9742 as “double-sided adhesive tape” of 0.5 mm thickness and 130 mm width. In a preferred embodiment the support lattice is comprised of a flexible, hydrophobic, liquid and gas impermeable material such as polyethylene; wherein the hydrophobicity of the surface adjacent to the absorbent material 107 provides further resistance to the transfer of liquid within absorbent material 107 to the surrounding flexible carrier lattice 104. More preferably the support lattice is composed of low density polyethylene film, with the side upon which adhesive layer 103 is interposed between the support lattice 102 and flexible carrier lattice 104, previously exposed to a low temperature corona discharge plasma, known in the art as “corona treatment”, so as to improve the adhesion of adhesive layers 103 and 110 and improve binding of the flexible carrier lattice to the support lattice, as well as improving adhesion of absorbent material 107. While the increase in polarization of the support lattice 102 would be expected to decrease the hydrophobicity and increase the hydrophilicity, which would give rise to increased wicking or fluid communication from the absorbent material 107 to outside the relevant test chamber; unexpectedly there is no observed leakage from within the test chamber to outside. The absorbent material 107 may be selected from any number of hypoallergenic materials known in the art to be capable of absorbing the liquid hapten of interest, by way of non-limiting example Whatman® Benchkote® Plus, sold by Merck KGAA, Darmstadt, Germany. In a preferred embodiment the absorbent material is polyethylene coated (one-side) absorbent material, sold by Ahlstrom GmbH (Baerenstein, Germany) as “LabSorb”, with the polyethylene coating adhered to the flexible lattice by way of interposed adhesive 110.

FIG. 2 presents an epicutaneous plaster 201, the epicutaneous patch formed by the placement of the flexible carrier lattice 206 over the support lattice 204. The alignment of a subset of holes in support lattice 204 with holes in flexible carrier lattice 206, results in holes 205 passing through the epicutaneous test plater 201.

Holes 205 provide improved flexibility to the epicutaneous test plaster and an outlet for sweat to evaporate, of particular utility when placed in contact with a patient's skin, maintaining contact of the hapten with the skin thus reducing or eliminating leaking of the haptens from the test chambers. Holes 205 also provide an opportunity to mark the underlying skin using a skin marker after the test patch is applied, with the resulting marks allowing for more accurate orientation of the location of the patch chamber positions after the patches are removed. These orientation marks may also assist in orienting patch chamber positions in cases where computer aided interpretation of patch test results are contemplated, for example based on digital photographs. The novel and advantageous incorporation of absorbent material 203, within test chambers 202, the absorbent material not in fluid communication with the surrounding flexible carrier lattice 206; enables the inclusion of liquid haptens and even more advantageously to the addition of liquid haptens to the test chambers, often referred to in the art as “loading” of a hapten, well in advance of the application of the epicutaneous test plaster to a patient, with the opportunity to subsequently seal the chambers to reduce or eliminate evaporation of the liquid.

As used in clinical practice, the epicutaneous test plaster is placed on the skin of a patient, with the chambers containing a hapten placed with the open side of the chamber against the skin, and the support lattice impermeable to liquid, forming the bottom of the chamber along with the absorbent material, opposing the open side of the chamber. Labelling of the side of the support lattice opposing the test chamber bottom layer is contemplated as part of the present invention in a preferred embodiment the labelling is with reversed text. The reversal of the text assists in the filling of the chambers, wherein the epicutaneous test plaster is oriented such that the bottom layer of the support lattice, which is impermeable to liquid, is placed on a flat surface, or in the sealing tool as further described herein. As such, the reversed labelling is viewed looking through the chamber opening, as being in the original “sense” orientation. This assists in the placement of haptens within the chamber, and proper documentation of the haptens in each chamber. While the absorbent material forming the bottom of the chamber may interfere with the visualization of the labelling, use of a light source underneath the epicutaneous test patch may assist in identification of specific chambers while loading; and subsequently following application to a patient.

Labels placed in the “sense” orientation (that is, not reversed) may be placed on the face of the support lattice opposing the test chamber bottom layer so as to allow easy identification of the chambers while the epicutaneous test plaster is on a patient.

Further optional labeling is contemplated by the present invention, by way of non-limiting example markings which provide means to determine the orientation of the epicutaneous test plaster while on a patient.

FIG. 3 shows an epicutaneous test patch of the present invention 301, containing test chambers 304, with absorbent material 303, a subset of holes 305 in the flexible carrier lattice 306 and support lattice 307; with removable cover layer 302 represented as partially removed from the epicutaneous test patch. After the patch is filled with haptens, removal of removable cover layer exposes the adhesive layer 105 (not shown in FIG. 3), the adhesive selected so as to be capable of reversibly adhering to either: 1) a continuous liquid and gas impermeable cover layer which itself is removable (as an intermediate step allowing storage and transportation prior to application to the skin of a patient), or 2) the skin of a patient for the duration desired for exposure to the hapten for the patient.

The prior art teaches that the loading of liquid haptens to test chambers in epicutaneous test patches for administration to the patient must occur within a short timeframe. For example the time between loading and application to skin must be no more than 3-5 minutes, so as to limit the opportunity for evaporation of the liquid hapten, or evaporation of the liquid in which the hapten is suspended or dissolved, or other loss of the hapten from the test chamber by diffusion such as is the case for formaldehyde which is a gas at room temperature. The prior art teaches a preferred mode of use where the liquid haptens are loaded into test chambers with the patient present, and then the loaded patches immediately applied to the skin. For use of the epicutaneous test plaster of the present invention in these use cases, the removal of the removable cover layer occurs prior to loading of the liquid haptens to the test chambers, followed by application of the epicutaneous test patch to the patient.

The prior art struggles with the effective transportation and storage of epicutaneous test plasters following loading, particularly when loading the chambers with liquid haptens. To address this limitation in the prior art, FIG. 4 shows an epicutaneous test patch of the present invention 401, containing test chambers 404, with absorbent material 403, a subset of holes 405 in the flexible carrier lattice 406 and support lattice 407; with a flexible, liquid and gas impermeable cover layer 402 represented as partially applied to the epicutaneous test patch. Where FIG. 3 presents the removal of the removable cover layer 302 which exposes the adhesive layer 105 (not shown in FIG. 3), flexible, liquid and gas impermeable cover layer 402 is applied after the patch has been loaded with liquid haptens. Liquid and gas impermeable cover layer 402 forms a gas and liquid impermeable seal over the test chamber previously loaded with liquid haptens. The liquid and gas impermeable cover layer 402 is presented as extending beyond the support lattice 407 and flexible carrier lattice 406, which provides improved grasping of layer 402 and its removal, from the support lattice prior to application of the epicutaneous test plaster to the skin of a patient.

With respect to liquid haptens maintained in a liquid state or dissolved in a liquid solvent; evaporation of the liquid has a negative impact on the ability to maintain contact of the hapten on the skin of a patient following administration. For those circumstances where storage or transportation of the epicutaneous test patch following loading of haptens is desired, the present invention advantageously contemplates the replacement of the removable cover layer with a flexible, liquid and gas impermeable cover layer, which forms a substantially air-tight seal over top of the flexible carrier lattice and test chambers, which may be assisted by the adhesive layer interposed between the flexible carrier lattice and the liquid and gas impermeable cover layer. This allows the loading of liquid haptens into the test chambers of the present invention, followed by the sealing of the test chambers by a flexible, liquid and gas impermeable cover layer; reducing or eliminating evaporation and other processes by which a liquid hapten may be lost from a test chamber.

The liquid and gas impermeable cover layer is chosen to provide both an effective seal over the individual test chambers, a durable adhesion to the flexible carrier lattice to maintain the seal during storage and shipping, and ease of removal from the flexible carrier lattice and interposed adhesive layer with limited reduction in the adhesive strength or quantity on the epicutaneous test patch. Ease of removal of the liquid and gas impermeable cover layer may be implemented by selection of adhesive or liquid and gas impermeable cover layer such that the strength of adhesion between the adhesive layer and flexible carrier lattice is greater than that between the adhesive layer and the liquid and gas impermeable cover layer. In a preferred embodiment, the liquid and gas impermeable cover layer is low-density polyethylene (LDPE) film, of thickness of between 0.002 inches (2 mil) to 0.03 inches (30 mil); and in an even more preferred embodiment between 2 mil and 20 mil. It is found that using the preferred embodiments and compositions described herein, the chambers will maintain a seal so as to prevent loss of a 2% formaldehyde solution, for up to 3 months.

The epicutaneous test plaster of the present invention is a significant advancement of the art, as it may reliably receive liquid or gel haptens and following sealing of the chamber as further described herein, provides for an epicutaneous test plaster, loaded with or without haptens; capable of long-term storage or shipping, for later application to a patient in need. This is in addition to the utility of the present invention for improved application of liquid haptens to the skin of a patient, whereby the novel design provides for reduction or elimination of communication of liquid haptens from one test chamber to adjacent test chamber.

FIG. 5 shows a cross section of an individual test chamber 501 of an epicutaneous test plaster of the present invention with a liquid and gas impermeable cover layer 506 in place, sealing the test chamber after a liquid hapten 511 has been added, thereby allowing for transportation and storage of the hapten loaded patch prior to application to the patient's skin. The exemplified epicutaneous test plaster is comprised of support lattice 502 which is a material impermeable to liquid and gas, and flexible carrier lattice 504 between adhesive layer 503 and 505. Removable cover layer, as presented in FIG. 1 as 106, is not shown in FIG. 5, instead liquid and gas impermeable cover layer 506 is shown, interacting with adhesive layer 505 and capable of forming a seal overtop test chamber 508. Test chamber 508 is formed by flexible carrier lattice 504 and adhesive layers 503 and 505; and is bounded on the bottom by support lattice 502. Within test chamber 508 is absorbent material 507, which is not in contact with the flexible carrier lattice 504; the separation represented by 509. Absorbent material 507 is adhered to the support lattice 502 by way of adhesive 510, by way of non-limiting example 3M 1524 medical grade adhesive. In a preferred embodiment the adhesive is provided as a layer contiguous with absorbent material 507, as presented in FIG. 5 as 510. Selection of adhesive used for adhering absorbent material 507 to the flexible carrier lattice is contemplated to be based upon the type of liquid being placed on the absorbent material, the composition of the support lattice, interactions with human skin, and composition of absorbent material; all within the capabilities of one skilled in the art. Also shown in FIG. 5 is the liquid hapten layer 511 which is absorbed into the absorbent material 507 and also present on the upper surface of absorbent material 507, is adjacent to, or in contact with, a patients skin upon its application as described herein.

The proximity of the liquid and gas impermeable cover layer above the absorbent material is a function of the height of the absorbent material relative to the flexible carrier layer. As the height of the absorbent layer increase, so does the volume of liquid it may absorb, with a consequence of decreased spacing between the top of the absorbent layer, and the proximal surface of the liquid and gas impermeable cover layer. This may give rise to contact between the absorbent layer and the liquid and gas impermeable cover layer.

This contact is of limited consequence for aqueous liquid haptens when polyethylene is used as the liquid and gas impermeable cover layer, with its high hydrophobicity, because very limited aqueous hapten remains on the liquid and gas impermeable cover layer when removed. Yet when nonpolar haptens are contained in the test chamber with polyethylene liquid and gas impermeable cover layer, or with aqueous haptens in the test chamber with a liquid and gas impermeable cover layer comprised of a more polar (or hydrophilic) material; the hapten may be transferred to the liquid and gas impermeable cover layer when it is removed from the carrier lattice.

Therefore, the present invention contemplates use of a liquid and gas impermeable cover layer which comprises an elevated portion over the test chambers formed in the epicutaneous patch described herein.

FIG. 9 shows a cross section of an embodiment of the individual test chamber 901 of an epicutaneous test plaster of the present invention. Removable cover layer, as presented in FIG. 1 as 106, is not shown in FIG. 9, instead liquid and gas impermeable cover 906 is shown, interacting with adhesive layer 905 and capable of forming a seal overtop test chamber 908. The exemplified epicutaneous test plaster is comprised of support lattice 902 which is a material impermeable to liquid and gas, and flexible carrier lattice 904 between adhesive layer 903 and 905. Test chamber 908 is formed by flexible carrier lattice 904 and adhesive layers 903 and 905; and is bounded on the bottom by support lattice 902. Within test chamber 908 is absorbent material 907, which is not in contact with the flexible carrier lattice 904; the separation represented by 909. Absorbent material 907 is adhered to the support lattice 902 by way of adhesive 910. The close proximity of liquid hapten 911 to liquid and gas impermeable cover layer 906 may give rise to contact during storage and transport.

FIG. 10 shows a cross section of an embodiment of the individual test chamber 1001 of an epicutaneous test plaster of the present invention. Removable cover layer, as presented in FIG. 1 as 106, is not shown in FIG. 10, instead liquid and gas impermeable cover 1006 is shown, extending above the plane of adhesive layer 1005 over the test chamber, creating increased space between the absorbent material 1007 and the liquid and gas impermeable cover layer 1006, which interacts with adhesive layer 1005 and can form a seal overtop test chamber 1008. An exemplary pyramidical shape is shown for the liquid and gas impermeable cover layer 1006, extending above the plane of the adhesive layer 1005. The present invention contemplates a number of shapes as possible, including generally convex, cubic, pyramidical, and other shapes capable of providing increased space between the liquid and gas impermeable layer and the absorbent material to which a liquid or gel hapten has been applied. The exemplified epicutaneous test plaster is comprised of support lattice 1002 which is a material impermeable to liquid and gas, and flexible carrier lattice 1004 between adhesive layer 1003 and 1005. Test chamber 1008 is formed by flexible carrier lattice 1004 and adhesive layers 1003 and 1005; and is bounded on the bottom by support lattice 1002. Within test chamber 1008 is absorbent material 1007, which is not in contact with the flexible carrier lattice 1004; the separation represented by 1009. Absorbent material 1007 is adhered to the support lattice 1002 by way of adhesive 1010. The extension of the liquid and gas impermeable cover layer above the test chamber provides further distance between the liquid or gel hapten 1011 and liquid and gas impermeable cover layer 1006, reducing contact between them during storage and transport.

### The Prior Art Provides Exemplary Materials Useful for Providing a Removable Liquid and

gas impermeable cover layer with an elevated portion located above the test chambers. In a preferred embodiment the removable liquid and gas impermeable cover layer would be 20 mil polystyrene laminated with a low-density polyethylene film layer, with the low density polyethylene layer placed closest to the adhesive layer.

FIG. 11 shows an epicutaneous test patch of the present invention 1101, containing test chambers 1104, with absorbent material 1103, a subset of holes 1105 in the flexible carrier lattice 1106 and support lattice 1107; with a flexible, liquid and gas impermeable cover layer 1102 represented as partially applied to the epicutaneous test patch. Where FIG. 3 presents the removal of the removable cover layer 302 which exposes the adhesive layer 105 (not shown in FIG. 3), flexible, liquid and gas impermeable cover layer 1102 is applied after the patch has been loaded with liquid or gel haptens, with an optional extension of the liquid and gas impermeable cover layer 1102 over the underlying flexible carrier lattice, assisting in its removal from the flexible carrier lattice, represented by 1108. The liquid and gas impermeable cover layer is represented without holes matching with the subset of holes 1105 in the flexible carrier lattice 1106. This provides an alternative embodiment of the present invention, where the liquid and gas impermeable cover layer provides additional support and stability to the epicutaneous patch following its sealing. The liquid and gas impermeable cover layer 1102 has an exemplary pyramidical shape extending above the plane of the adhesive layer 105 (not shown), providing increased space between the exposed surface of absorbent material 1103 and liquid and gas impermeable cover layer 1102. Liquid and gas impermeable cover layer 1102 forms a gas and liquid impermeable seal over the test chamber previously loaded with liquid haptens. The liquid and gas impermeable cover layer 1102 is presented as extending beyond the upper edge of the support lattice 1107 and flexible carrier lattice 1106, which provides improved grasping of layer 1102 and its removal, from the support lattice prior to application of the epicutaneous test plaster to the skin of a patient.

FIG. 6 presents a sealing tool 601 for use in the sealing of the individual test chambers of the epicutaneous patch disclosed herein. Sealing tool 601 is comprised of two parts, a lower portion 607 which is recessed so as to receive the epicutaneous patch of the present invention, the support lattice adjacent to the planar surface of the lower portion, and an upper portion 608, which protrudes an amount equivalent to the recession of the lower portion 607 and mirroring the shape of the epicutaneous test patch such that the lower portion 607 can receive the upper portion 608. Holes 602 in the lower portion 607, and holes 609 in the upper portion 608, are located so as to align above the test chambers in an epicutaneous test patch when placed within lower portion 607. The shape of the lower portion 607 and recession represented by 606, which is mirrored by the protrusion on upper portion 608 and represented by 605, may be selected so as to assist in the orientation and maintaining of the position of the epicutaneous patch when placed in the device (not shown). Holes 602 and 609 are of size and location such that the boundaries of each hole substantially align with the inner edge of the flexible carrier lattice that forms each of the test chambers. Optional hinges are presented in FIG. 6, with an inner cylindrical hinge element 604 attached to upper portion 608, rotating inside an annular hinge element 603 attached to lower portion 607. It is contemplated that the annular element may be attached to the upper portion and the cylindrical element attached to the lower portion, or any number of hinge elements as known in the art incorporated into the upper portion and lower portion so as to assist in the alignment and insertion of the protrusion of the upper element into the recession of the lower element.

FIG. 7 shows the sealing tool 701 in a fully opened state, ready to receive an epicutaneous test patch of the present invention, showing the lower portion 707 with recession 706, upper portion 708 with protrusion 705, and hinge elements 704 and 703. Holes 702, substantially align with the inner edge of the flexible carrier lattice that forms each of the test chambers are shown. It is contemplated that sealing tool 701 may be used for the loading of haptens into the test chambers of an epicutaneous test patch, which is placed in the lower portion 707, and maintained in place by way of recession 706 which corresponds to the shape of the epicutaneous test patch. With the epicutaneous test patch maintained in place in the lower portion 707, a user may more easily administer the hapten, liquid or otherwise, to the individual test chambers and absorbent material included therein. Following loading of the haptens, the test plaster may be removed, the removable cover layer removed, thereby exposing the adhesive layer, and the epicutaneous test patch then applied to the skin of a patient.

In circumstances where the epicutaneous test patch will be loaded, but not immediately used on a patient, it is contemplated that the removable cover layer may be removed, while maintained in the lower portion of the sealing tool; a liquid and gas impermeable cover layer placed over the entirety of the epicutaneous test patch and thereby in contact with the exposed adhesive layer, and the upper portion of the sealing tool then applied, with pressure, to the liquid and gas impermeable cover layer, with the protrusion from the upper portion of the sealing tool fitting within the recession of the lower portion of the sealing tool.

FIG. 8 shows the loading and sealing device 801 in a fully closed position and containing within it a sealed test patch loaded with liquid haptens, with upper portion 805 applied over the lower portion 806 of the sealing tool, with optional hinge elements 802 and 803 assisting in the alignment of the upper portion of the sealing tool with the lower portion of the sealing tool. 804 represents the upper surface of the liquid and gas impermeable cover layer exposed by holes 807 in upper portion 805. In a preferred embodiment, the liquid and gas impermeable layer is LDPE film of 2 mil thickness and flexible carrier lattice and adhesive layer formed by double-sided self-adhesive foam sold by Scapa group Plc, United Kingdom; with pressures applied to the upper portion of the sealing tool as low as 0.5 pounds per square inch sufficient to create substantial seals of the test chambers with the LDPE liquid and gas impermeable layer.

FIG. 12 shows the loading and sealing device 1201 in a fully closed position and containing within it a sealed test patch loaded with liquid haptens, with upper portion 1205 applied over the lower portion 1206 of the sealing tool, with optional hinge elements 1202 and 1203 assisting in the alignment of the upper portion of the sealing tool with the lower portion of the sealing tool. 1204 represents the upper surface of the liquid and gas impermeable cover layer in an exemplary pyramidical shape, exposed by holes 1207 in upper portion 1205.

The loading and sealing devices presented as 801 and 1201 may be used for the loading, and subsequent sealing of the epicutaneous patches of the present invention, which comprise a multiplicity of test chambers. The multiplicity of test chambers are presented as non-limiting examples in FIGS. 2, 3, 4, 6, 7, 8, 11 and 12 as having 40 test chambers. It is contemplated that following the loading of the test chambers in the loading and sealing devices, the epicutaneous patches may be further segmented using, for example, scissors; more preferably after loading and sealing with the liquid and gas impermeable cover layer. Further segmentation may be useful for the placement of the epicutaneous patches on patients, where less than the number of test chambers loaded are desired to be placed on a patient, or contiguously on a patient. The epicutaneous patch of the present invention as well as the loading and sealing device of the present invention, particularly when used together, provide substantial benefits to the healthcare professional loading and sealing the epicutaneous patch and the individual administering the epicutaneous patch to the patient.

While particular embodiments of the present invention have been described in the foregoing, it is to be understood that other embodiments are possible within the scope of the invention and are intended to be included herein. It will be clear to any person skilled in the art that modifications of and adjustments to this invention, not shown, are possible without departing from the spirit of the invention as demonstrated through the exemplary embodiments. The invention is therefore to be considered limited solely by the scope of the appended claims.

What is claimed is:

1. An epicutaneous test plaster comprising
a support lattice comprised of a material impermeable to liquid and gas containing a first multiplicity of holes,
a flexible carrier lattice containing a second multiplicity of holes,
a first adhesive layer for removable adhesion of the epicutaneous plaster to a skin portion,
a second adhesive layer for binding the support lattice to the flexible carrier lattice,
a liquid and gas impermeable cover layer extending over all the first adhesive layer of said flexible carrier lattice, and
a plurality of fluid absorbent material distributed over the support lattice;
wherein the second multiplicity of holes on the flexible carrier lattice is greater than the first multiplicity of holes on the support lattice;
wherein said flexible carrier lattice is adhered to the support lattice by way of the second adhesive layer;
wherein the plurality of test chambers are formed by way of second multiplicity of holes in the flexible carrier lattice describing a frame around a contiguous portion of the support lattice;
wherein a subset of the first multiplicity of holes on the support lattice align with the second multiplicity of holes in the flexible carrier lattice;
wherein the liquid and gas impermeable cover layer forms a seal over the top of the plurality of test chambers;
wherein the fluid absorbent material is located within said plurality of test chambers, said fluid absorbent material not in contact with either of said flexible carrier lattice or liquid and gas impermeable cover layer; and
wherein the liquid and gas impermeable cover layer is removably secured to the flexible carrier lattice by way of said first adhesive layer.

2. The epicutaneous test plaster of claim 1 wherein the flexible carrier lattice is comprised of polyethylene foam and said first and second adhesive layers are comprised of a medical grade adhesive.

3. The epicutaneous test plaster of claim 2 wherein the polyethylene foam has a thickness of between 0.4 mm and 0.6 mm.

4. The epicutaneous test plaster of claim 1 wherein said liquid and gas impermeable layer is a lattice with a third multiplicity of holes.

5. The epicutaneous test plaster of claim 4 wherein said third multiplicity of holes align with the second multiplicity of holes in the flexible carrier lattice.

6. The epicutaneous test plaster of claim 1 wherein at least one unique chamber identifier is printed on the material impermeable to liquid and gas proximate to a test chamber.

7. The epicutaneous test plaster of claim 1 wherein the liquid and gas impermeable cover layer is comprised of a low density polyethylene film of 2 mil thickness.

8. The epicutaneous test plaster of claim 1 wherein the liquid and gas impermeable cover layer extends above the plane of the first adhesive layer over the test chamber.

9. A sealing tool for use in sealing an epicutaneous test plaster of claim 1 the sealing tool comprising
a lower portion with a multiplicity of lower openings, and
an upper portion with a multiplicity of upper openings;
wherein said lower portion is recessed so as to receive said epicutaneous patch;
wherein said upper portion protrudes an amount equivalent to the recession of said lower portion;
wherein both upper portion and lower portion mirror the shape of the epicutaneous test patch such that said lower portion can receive said upper portion;
and wherein lower portion lower openings and upper portion upper openings are positioned such that when said epicutaneous test patch is placed within said lower portion the chambers in said epicutaneous patch are aligned with both the upper portion upper openings and lower portion lower openings.

10. The sealing tool of claim 9 wherein the upper portion and lower portion are in mechanical communication by way of a hinge.

11. An epicutaneous test plaster comprising
a support lattice comprised of a material impermeable to liquid and gas containing a first multiplicity of holes,
a flexible carrier lattice containing a second multiplicity of holes,
a first adhesive layer for removable adhesion of the epicutaneous plaster to a skin portion,
a second adhesive layer for binding the support lattice to the flexible carrier lattice,
a liquid and gas impermeable cover layer extending over all the first adhesive layer of said flexible carrier lattice, and
a plurality of fluid absorbent material distributed over the support lattice;
wherein the second multiplicity of holes on the flexible carrier lattice is greater than the first multiplicity of holes on the support lattice;
wherein said flexible carrier lattice is adhered to the support lattice by way of the second adhesive layer;
wherein the plurality of test chambers are formed by way of second multiplicity of holes in the flexible carrier lattice describing a frame around a contiguous portion of the support lattice;
wherein a subset of the first multiplicity of holes on the support lattice align with the second multiplicity of holes in the flexible carrier lattice;
wherein said fluid absorbent material not in contact with said flexible carrier lattice;
wherein the liquid and gas impermeable cover layer forms a seal over the top of the plurality of test chambers;
wherein the fluid absorbent material is located within said plurality of test chambers; and
wherein the liquid and gas impermeable cover layer is removably secured to the flexible carrier lattice by way of said first adhesive layer.

12. The epicutaneous test plaster of claim 11 wherein the flexible carrier lattice is comprised of polyethylene foam and said first and second adhesive layers are comprised of a medical grade adhesive.

13. The epicutaneous test plaster of claim 12 wherein the polyethylene foam has a thickness of between 0.4 mm and 0.6 mm.

14. The epicutaneous test plaster of claim 11 wherein said liquid and gas impermeable layer is a lattice with a third multiplicity of holes.

15. The epicutaneous test plaster of claim 14 wherein said third multiplicity of holes align with the second multiplicity of holes in the flexible carrier lattice.

16. The epicutaneous test plaster of claim 11 wherein at least one unique chamber identifier is printed on the material impermeable to liquid and gas proximate to a test chamber.

17. The epicutaneous test plaster of claim 11 wherein the liquid and gas impermeable cover layer is comprised of a low-density polyethylene film of 2 mil thickness.
